(19) United States
(12) Patent Application Publication
(10) Pub. No.: US 2005/0142234 A1
(43) Pub. Date: Jun. 30, 2005

(54) Pharmaceutical activity and manufaturing method of the four-plant compound immunoenhanceers

(76) Inventors: Jiao Gong, Arcadia, CA (US); Wei Jiang, Arcadia, CA (US)

Correspondence Address: JIAO GONG, 411 S. 3RD AVE #B, ARCADIA, CA, 91006, US

(21) Appl. No.: 10/745,816

(22) Filed: Dec. 29, 2003

(51) Int. Cl. A61K 35/78

(52) U.S. Cl. 424/773; 424/777

(57) ABSTRACT

The injection or oral administration of exogenous IL-2 can damage the original functional equilibrium in the immune system of the body itself while modulating and enhancing immune function. The four-plant compound immunoenhancers differ from the exogenous input of IL-2 preparation. The compound products can promote the formation of endogenous IL-2 in the body, regulate and enhance the immune system. It is of greater clinical significance than the supplementation of exogenous IL-2 preparation. The four-plant ingredients are absorbed, eluted, isolated and purified with WLD resin in the rigid and delicate conditions. The quantitative analysis by finger atlas with a high performance liquid chromatograph ensures the steady and uniform quality of products. The experiment on pharmacological activity shows the compound products can facilitate the formation of spleen lymphocyte IL-2 remarkably.

The pharmacological activity and manufacturing process of the four-plant compound immunoenhancers. The four plants include Radix Rehmanniae, Fructus, Corni, Fructus Lycii and Radix Angelicae Sinsensis.

### BACKGROUND OF THE INVENTION

The pharmacological activity and manufacturing process of the four-plant compound immunoenhancers belong to the combined patent concerning the pharmacological activity of natural products and methods of isolating and purifying natural products in the U.S. patents. The four-plant compound products are immunomodulators with the pharmacological activity that remarkably promotes the formation of mouse spleen lymphocyte IL-2.

In 1958 the U.S. No. 4 Federal circuit court stated in the first case involving a naturally existing compound—vitamin B12, when a product of nature was a new and useful synthetic substance, there was not regulation on the exclusion of the grant of a patent in the Patent Bill (1952). In terms of raw materials supplied by Nature, all the real things protected by patent are products of nature. The court further pointed out the fact that a new and useful product was obtained by separation, concentration and purification of natural raw materials didn't impair its patentability. The pharmacological activity and manufacturing process of the four-plant compound immunoenhancers are in conformity with the requirement of patentability by the U.S. No. 4 Federal Circuit Court.

Drugs that can specifically enhance or regulate body immune function mainly cover immunological adjuvants, immuno-restoration agents, immuno-substitutes and immunomodulators. Especially, the immunomodulator is most promising because it plays a two-way regulatory role in the immune function and can regulate excessively high or low immune function to normal level without any effect upon normal immune function. Some immunomodulators have been used clinically in the treatment of infectious diseases and rheumatism as well as in the joint chemotherapy of tumors. As immune system and modulation are understood more profoundly and continuously, some substances that were thought to have little to do with immunoreaction are found to be immunomodulators. Some immunosuppresants can't be changed into immunoenhancers in some cases. So immunomodulators have a wide scope and chiefly consist of cytokines, which are used as imunomodulators for bions and plants, antibody and chemical synthetic drugs. Typical of them is interleukin 2, which is a glucoprotein secreted by activated T_{B} cell and macrophage. IL-2 acts by combination with IL-2 receptor. Its main functions are to activate B cells to proliferate, disintegrate and generate antibodies for promoting the proliferation and disintegration of T cells, activate macrophages, enhance the activity of killer cell LAK activated by NK cell and lymphokine and induce other cytokines to give rise to more extensive immunoenhancement and immunomodulation. The oral administration and injection of exogenous IL-2 can also damage the original function and equilibrium of the immune system in the body while modulating and enhancing immune function. The four-plant compound immunoenhancers can trigger the change of IL-2 in the body itself without any damage to the function and equilibrium of the immune system in the body.

### BRIEF SUMMARY OF THE INVENTION

Enhanced T cell function is of great clinical significance. The formation of and increase in interleukin 2 is one of the major indications. IL-2 is an important cytokine that involves in immune response and modulation. In the presence of mitogen, IL-2 can act upon mouse thymocyte and induce its proliferation. The activity of IL-2 is revealed in terms of the amount of ³H-TdR blended in the proliferation of thymocyte to observe the effect of the compound products on the formation of mouse lyphocyte IL-2. The experimental result shows that the four-plant compound immunoenhancers can remarkably induce the proliferation of mouse thymocyte with significant difference compared with corresponding control group. This indicates the compound products can facilitate the formation of mouse spleen lymphocyte IL-2. The four-plant compound immunoenhancers differ from the exogenous input of IL-2 preparation in terms of the formation of mouse spleen lymphocyte IL-2. The compound products can promote the formation of endogenous IL-2 in the body to modulate and enhance the immune system. Its clinical implication is greater than the supplementation of exogenous IL-2 preparation as a more excellent immunomodulator.

### BRIEF DESCRIPTION OF THE SEVERAL VIEWS OF THE DRAWING

TABLE 1
Material Source and Composition of The pharmacological activity and
manufacturing process of the four-plant compound immunoenhancers

| | | |
|---|---|---|
| 1. | Radix Rehmanniae | 33% |
| | Surophulariaceae | |
| | Root of Rehmannia Glutinosa Libosch. | |
| 2. | Fructus Corni | 20% |
| | Cornaceae | |
| | Fruit of Cornus Offcinalis Sieb. et Zuce | |
| 3. | Fructus Lycii | 20% |
| | Solanaceae | |
| | Fruit of Lycium BarbarumL. | |
| 4. | Radix Angelicae Sinensis | 27% |
| | Umbelliferae | |
| | Root of Angelica Sinensis (Dliv.) Diels | |
| | Total | 100% |

TABLE 2
Manufacturing Method and Flowchart for the
Four-plant Compound Immunoenhancers

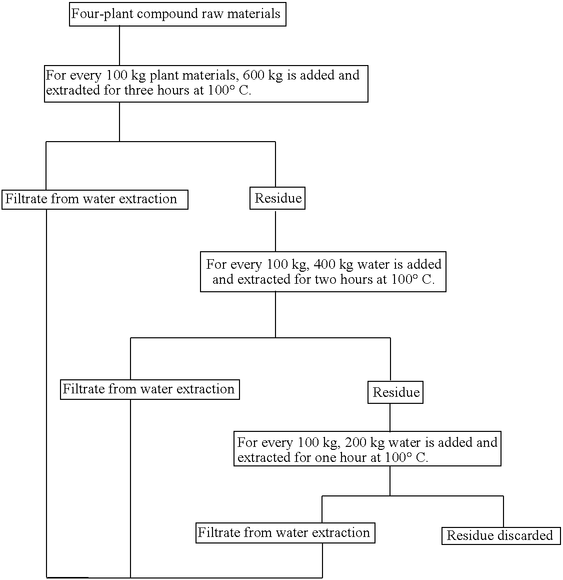

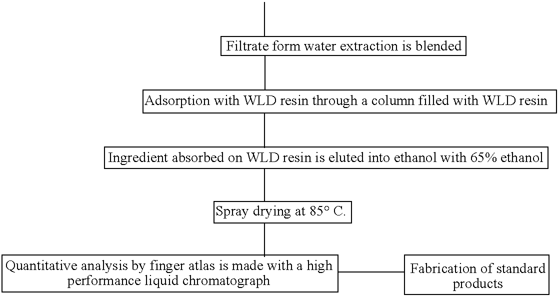

Table 3 Pharmacological Activity of the Four-plant Compound Immunoenhancers

1. Effect of the hour plant compound imunoenhancers (referred to as compound products for short) upon the formation of normal mouse interleukin 2.

(1) Mice are randomly allotted to two groups, compound products group and control group. They are perfused with compound products and water respectively, twice a day for 0.6 ml each time in a consecutive period of four weeks.

(2) Formation of IL-2: Bloodletting is done by picking off the eye ball of mouse, which is killed by cutting the neck and dipped in 75% ethanol for disinfection for five minutes. The abdominal cavity is cut open under sterile conditions and the spleen is taken out. Then it is placed in a plate filled with 5% calf serum and 1% double resistant Hank's fluid and ground on the stainless steel wire of 100 holes. Suspension of individual spleen cell is obtained after filtering through a single-layer nylon screen. It is rinced twice with 5% calf serum Hank's liquor, namely, it is centrifugalized twice at 1000 rmp, ten minutes for each and live cell count is taken. After the second centrifugation, the supernatant fluid is discarded. A suspension of 5×10⁶/ml spleen cells is made up with pure RPMI-1640 medium and ConA is added to achieve the final concentration of 5 μg/ml. Then it is put into the cell culture flask and heated for 24 hours in the culture box of 5% CO₂ at 37° C. The supernatant fluid is reaped by centrifugation for 20 minutes at 2500 rmp. After disinfection through 0.22 μm filter, it is kept in a refrigerator at 20° C. below zero.

(3) Detection of IL-2 activity: (1) preparation of mouse thymocyte suspension: 6-8 week old male mouse of C₅₇BL/6 is taken and killed by bloodletting from eye ball. The abdominal cavity is opened under sterile conditions and thymus is removed. It is rinsed with Hank's fluid containing 5% calf serum and transferred to a plate of Hank's fluid containing a small amount of 5% calf serum and ground on the stainless steel wire. Suspension of thymocyte is obtained by filtering through a single-layer nylon screen. The suspension is rinsed twice with Hank's fluid containing 5% calf serum, centrifugalized for 10 minutes at 1000 rmp each time and live cell count is taken. After the second centrifugation, the supernatant fluid is discarded and a suspension of 1.0×10⁷/ml thymocyte is made up with PRMI-1640 culture medium. (2) cell culture: Samples of IL-2 are diluted in a ratio of 1/1, 1/2, 1/4, 1/8, 1/16 and 1/32 into six aliquots and placed in a 96-hole flat-bottom culture plate with 100 ul for each hole. Three parallel holes are made for each dilution. To each hole are added 100 μl of thymocyted suspension and 20 μl of 30 μg/ml ConA fluid. Simultaneously, corresponding trial ConA control and culture medium control are arranged. The culture plate is incubated in the culture box of 5% CO₂ for 72 hours at 37° C. After 66 hours, to each hole is added 20 μl ³H-TdR (0.2 μCi) and cultured for another 6 hours. Cells are collected on the glass fiber filter paper with multi-sucker cell sample collector after drying, value of ³H-TdR cpm incorporating DNA is measured using a liquid scintillation counter.

[Result] for mice of compound products group, their supernatant fluids of spleen lymphocyte culture diluted in a ratio of 1/2, 1/4, 1/8, 1/16 and 1/32 can induce the proliferation of mouse thymocyte remarkable with significant difference compared with the control group, which indicates the compound products can promote the formation of mouse spleen lymphocyte noticeably (Table 1)

TABLE 1
Effect of Compound Products upon the Formation of Normal Mouse
Interleukin-2 (IL-2) (cmp, X ± SD)

| | Groups | | | |
|---|---|---|---|---|
| | | Animal | Compound | Animal |
| Dilution | Control group | No. (n) | products group | No. (n) |
| 1:2 | 14484 ± 3628 | 5 | 22644 ± 4658*** | 5 |
| 1:4 | 12368 ± 3504 | 5 | 17092 ± 2431** | 5 |
| 1:8 | 9255 ± 2565 | 6 | 14297 ± 2096*** | 5 |
| 1:16 | 6809 ± 1625 | 5 | 9688 ± 1448*** | 6 |
| 1:32 | 4853 ± 1103 | 6 | 6119 ± 812** | 5 |
| 1:64 | 4570 ± 224 | 6 | 4459 ± 461* | 6 |
| ConA | 3060.0 ± 459.7 | Cells | 80.7 ± 10.8 | |
| compar- | | comparison | | |
| ison | | | | |

### DETAILED DESCRIPTION OF THE INVENTION

1. The pharmacological activity and manufacturing process of the four-plant compound immunoenhancers belong to the combined patent concerning the pharmacological activity of natural products and methods of isolating and purifying natural products. The four-plant compound products are composed of 33% Radix Rehmanniae, 20% Fructus Corni, 20% Frudus Lycii and 27% Radix Angelicae Sinsensis.

2. To the four-plant compound materials are added 600 kg, 400 kg and 200 kg of water for every 100 kg and the materials are extracted for 3, 2 and 1 hours at 100° C. The filtrate from water extraction is blended and absorbed with WLD resin through the column filled with WLD resin. The ingredient absorbed on WLD resin is eluted with 65% ethanol and spray dried at 85° C. Standard products are made after quantitative analysis by finger atlas with a high performance liquid chromatograph.

3. Effect of the four-plant compound immunoenhancers on the formation of normal mouse interleukin 2. The experimental result of pharmacological activity shows that for mice of compound products group, their supernatant fluids of spleen lymphocyte culture diluted in a ratio of 1/2, 1/4, 1/8, 1/16 and 1/32 can induce the proliferation of mouse thymocyte remarkably, indicating the compound products can facilitate the formation of mouse spleen lymphocyte noticeably.

1. Now I declare that the pharmacological activity and manufacturing process of the four-plant compound immunoenhancers, namely, Radix Rehmanniae. Fructus Corni, Fructus Lycii, Radix Angelicae Sinsensis are my invention.
I think my invention covers the products made by extracting with water the four-plant compound materials, absorbing with WLD resin, eluting, drying and quantitative analysis.
I think the four-plant compound immunoenhancers of my invention feature the pharmacological activity of facilitating the formation of spleen lymphocyte IL-2 remarkably.
